(19) United States
(12) Patent Application Publication
(10) Pub. No.: US 2004/0139962 A1
(43) Pub. Date: Jul. 22, 2004

(54) John McArthur Bailey memorial synthetic sugar and fossil fuels (synthetic oil) process

(76) Inventor: Robert Texas Bailey, Alpine, TX (US)

Correspondence Address: Robert T. Bailey, P.O. Box 9740, Alpine, TX, 79831, US

(21) Appl. No.: 10/270,767

(22) Filed: Jan. 22, 2003

(51) Int. Cl. C13K 1/00; C07C 1/00; C10L 7/00

(52) U.S. Cl. 127/30; 204/157.15; 127/42; 44/300; 44/265

(57) ABSTRACT

1. A particular solution must be made that by removing both Oxygen and 2 Hydrogen will form what you want. You may have to hydrogenate double bonds so you can keep removing one oxygen and 2 hydrogen(keep Hydrogen, reform). You may want to use Platinumn.

2.The solution is put in microwave oven to take the place of currents of electrodes(small particles of iron in solution). You can increase the effect by adding powdered iron to solution! The microwave oven produces currents in particular solution, removing one oxygen and two hydrogen.

3. Example: Put carbonated water in microwave oven for 5 minutes it will form sugar and have a sweet taste.

4. Example: Put carbonated water and bubble methane through solution, and it will form gasoline.

5. This process could be used to make many chemicals.

When I was young my older brother Johnny showed me how to separate water into oxygen and hydrogen by using a 9 volt battery with two wires in water and covered the wires with aluminum foil and put salt in water. He put two bottles completely in water and lifted the bottles out of except the mouth of bottles and mounted them in that position and put one of the aluminum foil covered under each bottle, and in less than week one bottle was full of Hydrogen and the other with about half as much was full of Oxygen.

He removed the Oxygen bottle and quickly turned it right side up and carried it as Oxygen was heaver than air and would stay in bottle which it did, and he set on table. He lit on fire a long wood match diameter stick and then blew the fire out and with only a red glow of smoking ember he stuck the ember in the Oxygen bottle and it relit on fire proving that there was Oxygen in the bottle!

Then he removed the hydrogen bottle but carried it upside down as Hydrogen is lighter than air. He lit a match and placed it by the lip of jar and turned the bottle as to poor upside down, when the Hydrogen reached the match there was a large poof as the Hydrogen lit!

What I am trying to do is turn Carbon Dioxide and water to Sugar and Oxygen but found that 3CO₂+2H₂+C₃H₈=C₆O₆H₁₂ is possible, in a few steps.

(Carbon Dioxide-Hydrogenation)

Thermodynamics Entropy: When they first started studying chemical reactions they figured chemical reactions will go in the direction that would release heat (gas will bum to carbon dioxide and water, carbon dioxide and water will not turn to gas naturally). But then they found some chemical reactions that get colder rather than release heat. So they came up with Entropy(Symbol=S). It is based oh statistics. It can also be calculated from Heat capacity of a chemical, by dividing the heat capacity of the chemical, every degree from absolute zero to the temperature you are at but normally considered at 25 degrees C., and sum the results! Entropy always increases and never decreases. So if you calculate the entropy of gas and oxygen and then carbon dioxide and water, you find carbon dioxide and water has a higher entropy, so the chemical reaction will naturally go in the direction of gas and oxygen to carbon dioxide and water.

What I am trying to do is turn Carbon Dioxide and water to Sugar and Oxygen but found that 3CO₂+2H₂+C₃H₈=C₆O₆H₁₂ is possible!

Looking at the equation:

C₆O₈H₁₂+6O₂=6CO₂+6H₂O

Since Entropy only increases and never decreases, 6CO₂+6H₂O must have a higher Entropy than C₆O₆H₁₂+6O₂, and since by adding sun light(heat) to 6CO₂+6H₂O will increase its temperature and Entropy and would make it even harder for it to go to lower Entropy of C₆O₆H₁₂+6O₂. Because of this I have all ways argued that Sun light is not added to 6CO₂+6H₂O to make C₆O₆H₁₂+6O₂, but that Sun light is used to make electricity(photoelectric effect).

Now that electricity could be used to make H₂ and O₂ by letting the current flow through water, and the gases could be used to drive a chemical reaction in the direction of sugar from carbon dioxide

The chemical reaction C₆O₆H₁₂+6O₂=6CO₂+6H₂O will go in the direction of sugar and oxygen to carbon dioxide and water. We know the Entropy of each gas, C₆O₆H₁₂+6O₂ (6×230)=6CO_{2(6×210)}+6H₂O (6×180) leaving sugar with a Entropy that must be less than or About 1000.

Keeping in mind the high heat capacity of water and also its high Entropy because of this and the chemical reaction goes in the direction that has water, so lets see If we can get water on the product of chemical reaction and get the Entropy to be more on product side or close to that.

Now to reverse this process, to go from carbon dioxide and water to sugar and oxygen but lets change and use the water to be separated into H2 and O2 and then add H2 to CO2, lets look at this equation:

4H₂+CO₂=CH₄+2H₂O The Entropy 4H₂+CO₂ (About 700J/K)=Entropy CH₄+2H₂O(About 600J/K) Trying to go from Entropy 4H₂+CO₂ (About 700/K)-To-Entropy CH₄+2H₂O(About 600J/K)

there is a decrease of Entropy of about 100 and a chemical reaction will go in the direction that Entropy will increase and there is two ways to accomplish this.

One way is to decrease the Entropy of 4H₂+CO₂ (About 700J/K) by adding pressure to it giving it a lower Entropy than CH₄+2H₂O(About 600J/K). Now looking at this equation there is 5 moles on one side of equation and 3 moles on the other(product side) so by doing work(adding pressure to product side you could increase Entropy to product side and make the chemical reaction go in the direction of the product, and doing this in normal pressure of one atmosphere work will be done and could make reaction go forward to product side).

The other way is to increase the temperature as you look at the heat capacities(symbol Cₚ) of reactance and the products and we know that Entropy can be calculated by dividing the heat capacity per degree Kelvin from absolute zero to temperature you are at. Now if you have something that has high heat capacity like water(Cₚ=75) its Entropy will increase faster than Co₂(Cₚ=37), So lets look at the equation again Entropy 4H₂+CO₂ (About 700J/K)=Entropy CH₄+2H₂O(About 600J/K) and lets look at the heat capacity of each 4H₂ (Cₚ=29 per mole or 116)+CO₂ (Cₚ=37) and CH₄ (Cₚ=36)+2H₂O(Cₚ=75 per mole or 150). Giving us 116+37=153 on one side of equation (reactance)and 36+150=186 on the other(products). So as you increase the temperature the products will gain in entropy and when the products have more Entropy than reactance the chemical reaction will go forward.

The simplest way to get Carbon Dioxide—Hydrogenation is to place carbonated water in a microwave and turn it on for a few minutes. The natural Ph in the water with the carbon dioxide in bubbles and heat in microwave will form trace amount of sugar.

Also

So in a pan of Carbonated water I put a plastic bottle in water and then lifted the bottle out of water except opening of bottle and with tape mounted to solid surface, holding bottle above water and leaving mouth of bottle in water. Now I got 30 volt power supply and ran two wires(Platinum) into water from each electrode and the one that produced the most bubbles(this is Hydrogen) I let bubble in the bottle and with carbonation bubbling from water, filled the bottle just before it stopped bubbling. After a few days of letting hydrogen bubble into CO₂ the gas in the bottle became less and adding more carbonated water to let carbonation refill bottle then letting hydrogen again many times reduce the gas, I added half as much propane to the gas mixture from lighter to the gas and platinum wire with no charge only as catalyst to gas and solution, after a day I got a blackish material on sides of pan!

Now looking at this equation, 2H₂+CO₂+CH₄=C₂H₆+2H₂O because to link carbons together it may take two different kinds of carbons. I dried some of the blackish material and lit it with lighter and it burned like an ember(glow red and as it went out a small puff of smoke) Thinking if I oxidized this blackish material a little I may get sugar I left in sun light with water and got a white material as water dried. Re adding water the white material dissolved like sugar, more test need to be done, and is their may be a way to make sugar directly.

6H₂+18CO₂=C₆O₆H₁₂+6H₂O

The equation 3H₂+CO₂+CH₄=C₂+C₆+H₂O and its Entropy, 3H₂+CO₂+CH₄ (About 750)=C₂+C₆+H₂O(About 625)

6H₂+18CO₂=C₆O₆H₁₂+6H₂O

What I am trying to do is turn Carbon Dioxide and water to Sugar and Oxygen but found that 3CO₂+2H₂+C₃H₈=C₆O₆H₁₂ is possible!

### SUMMARY OF INVENTION

They have been trying to turn carbon dioxide and water to sugar and oxygen like plant but what I found was what it is possible to make carbon dioxide and hydrogen react. By adding Propane to reactance I was able to get solid oil like material for product. By light oxidation I was able to get a sugar like material.

1. I can make synthetic sugar, and synthetic fossil fuels.

2. I can make synthetic sugar.

3. I can make a gasoline that is pure and has less pollution than natural gasoline when burned.

4. I have a process using a particular solution, and using microwave that can form many chemicals.
